(19) United States
(12) Patent Application Publication
(10) Pub. No.: US 2022/0047246 A1
(43) Pub. Date: Feb. 17, 2022

(54) METHOD AND DEVICE FOR EVALUATING MUSCLE TENSION

(71) Applicant: SHENZHEN INSTITUTES OF ADVANCED TECHNOLOGY, Shenzhen, Guangdong (CN)

(72) Inventors: Yang XIAO, Shenzhen, Guangdong (CN); Hairong ZHENG, Shenzhen, Guangdong (CN); Teng MA, Shenzhen, Guangdong (CN); Congzhi WANG, Shenzhen, Guangdong (CN); Fei LI, Shenzhen, Guangdong (CN)

(21) Appl. No.: 17/298,571

(22) PCT Filed: Dec. 14, 2018
(86) PCT No.: PCT/CN2018/121295

§ 371 Date: May 29, 2021

(51) Int. Cl. A61B 8/08; A61B 8/00

(52) U.S. Cl. CPC A61B 8/485; A61B 8/08; A61B 8/463

(57) ABSTRACT

A method and device for evaluating muscle tension, the method comprising: obtaining the elastic modulus values of the measured skeletal muscle under different joint angles and each of the joint angles (S11); determining the changing trend of the elastic modulus value of the measured skeletal muscle according to the elastic modulus values of the measured skeletal muscle under different joint angles and each of the joint angles (S12); and evaluating the muscle tension level of the measured skeletal muscle according to the changing trend of the elastic modulus value of the measured skeletal muscle (S13). The method can be used to evaluate the muscle tension level.

### CROSS-REFERENCE TO RELATED APPLICATION

This Application is a national stage application of PCT/CN2018/121295. This application claims priority from PCT Application No. PCT/CN2018/121295, filed Dec. 14, 2018, the content of which is incorporated herein in the entirety by reference.

### FIELD OF THE INVENTION

This application relates to the field of medical technology, in particular to a method and device for evaluating muscle tension.

### BACKGROUND TECHNOLOGY

Muscle tension is the force generated by muscle cells pulling each other, which can also be understood as the tensity of muscles in a static and relaxed state. Moreover, muscle tension is the basis for maintaining various postures and regular movements of the body.

As muscle tension is the basis for maintaining various postures and regular movements of the body, the evaluation of muscle tension of muscle tissues has important guiding significance for the diagnosis and monitoring of diseases, the formulation of later rehabilitation plans, and the prognosis of tissue function, etc. However, how to evaluate muscle tension has become a problem.

### SUMMARY OF THE INVENTION

In order to solve the above technical problems, the embodiments of the present application provide a method and device for evaluating muscle tension to achieve the purpose of evaluating muscle tension levels. The technical solutions are as follows:

A method for evaluating muscle tension, comprising:

obtaining the elastic modulus values of the measured skeletal muscle under different joint angles and each of the joint angles;

determining the changing trend of the elastic modulus value of the measured skeletal muscle according to the elastic modulus values of the measured skeletal muscle under different joint angles and each of the joint angles;

and evaluating the muscle tension level of the measured skeletal muscle according to the changing trend of the elastic modulus value of the measured skeletal muscle.

Preferably, obtaining the elastic modulus values of the measured skeletal muscle under different joint angles comprises: obtaining the elastic modulus values of the measured skeletal muscle under each of the joint angles for multiple measurements; performing an average calculation on the elastic modulus values of the measured skeletal muscle under each of the joint angles for multiple measurements, respectively, and using the result of the average calculation as the elastic modulus value of the measured skeletal muscle under the joint angle.

Preferably, the elastic modulus values of the measured skeletal muscle under different joint angles are measured by an ultrasonic diagnostic machine.

Preferably, the elastic modulus values of the measured skeletal muscle under different joint angles are: the elastic modulus values of the thickest position of muscle belly of the measured skeletal muscle under different joint angles measured by the ultrasonic diagnostic machine.

Preferably, determining the changing trend of the elastic modulus value of the measured skeletal muscle according to the elastic modulus values of the measured skeletal muscle under different joint angles and each of the joint angles comprises:

plotting a changing trend graph of the elastic modulus value of the measured skeletal muscle with each of the joint angles as the abscissa and the elastic modulus values of the measured skeletal muscle under different joint angles as the ordinate;

and the changing trend graph is used to characterize the changing trend of the elastic modulus value of the measured skeletal muscle.

A device for evaluating muscle tension, comprising:

an obtaining module, configured to obtain the elastic modulus values of the measured skeletal muscle under different joint angles and each of the joint angles;

a determining module, configured to determine the changing trend of the elastic modulus value of the measured skeletal muscle according to the elastic modulus values of the measured skeletal muscle under different joint angles and each of the joint angles;

and an evaluating module, configured to evaluate the muscle tension level of the measured skeletal muscle according to the changing trend of the elastic modulus value of the measured skeletal muscle.

Preferably, the obtaining module comprises:

an obtaining submodule, configured to obtain the elastic modulus values of the measured skeletal muscle under each of the joint angles for multiple measurements;

and a determining submodule, configured to perform an average calculation on the elastic modulus values of the measured skeletal muscle under each of the joint angles for multiple measurements, respectively, and use the result of the average calculation as the elastic modulus value of the measured skeletal muscle under the joint angle.

Preferably, the elastic modulus values of the measured skeletal muscle under different joint angles are measured by an ultrasonic diagnostic machine.

Preferably, the elastic modulus values of the measured skeletal muscle under different joint angles are: the elastic modulus values of the thickest position of muscle belly of the measured skeletal muscle under different joint angles measured by the ultrasonic diagnostic machine.

Preferably, the determining module comprises:

a plotting module, configured to plot a changing trend graph of the elastic modulus value of the measured skeletal muscle with each of the joint angles as the abscissa and the elastic modulus values of the measured skeletal muscle under different joint angles as the ordinate;

and the changing trend graph is used to characterize the changing trend of the elastic modulus value of the measured skeletal muscle.

Compared with the prior art, the application has the beneficial effects as follows:

In the application, the elastic modulus values of the measured skeletal muscle under different joint angles are obtained as the basis for monitoring the muscle tension of the measured skeletal muscles; determining the changing trend of the elastic modulus value of the measured skeletal muscle according to the elastic modulus values of the measured skeletal muscle under different joint angles and each of the joint angles, and evaluating the muscle tension level of the measured skeletal muscle according to the changing trend of the elastic modulus value of the measured skeletal muscle, based on the principle that the change of the elastic modulus value can characterize the change of resistance during skeletal muscle activity, and the change of resistance can reflect the situation of muscle tension.

### BRIEF DESCRIPTION OF THE DRAWINGS

In order to explain the technical solutions in the embodiments of the application more clearly, the following will briefly introduce the drawings needed in the description of the embodiments. Obviously, the drawings in the following description are only for some embodiments of the application. Other drawings can be obtained for those of ordinary skill in the art based on these drawings without creative work.

FIG. 1 is a flow chart of the method for evaluating muscle tension provided by the application;

FIG. 2 is a schematic diagram of the protractor provided by the application for measuring the joint angle;

FIG. 3 is a schematic diagram of measuring the elastic modulus value in the region of interest provided by the application;

FIG. 4(a)-(f) shows the different changing trends of the elastic modulus value of the measured skeletal muscle;

FIG. 5 is a schematic diagram of a logical structure of the device for evaluating muscle tension provided by the application.

### DETAILED DESCRIPTION OF THE INVENTION

The technical solutions in the embodiments of the application will be clearly and completely described below in conjunction with the drawings in the embodiments of the application. Obviously, the described embodiments are only a part of the embodiments of the application, rather than all the embodiments. Based on the embodiments in the application, all other embodiments obtained by those of ordinary skill in the art without creative work shall fall within the protection scope of the application.

The embodiment of the application discloses a method for evaluating muscle tension, comprising: obtaining the elastic modulus values of the measured skeletal muscle under different joint angles; determining the changing trend of the elastic modulus value of the measured skeletal muscle according to the elastic modulus values of the measured skeletal muscle under different joint angles and each of the joint angles; and evaluating the muscle tension level of the measured skeletal muscle according to the changing trend of the elastic modulus value of the measured skeletal muscle. In the application, the evaluation of muscle tension can be achieved.

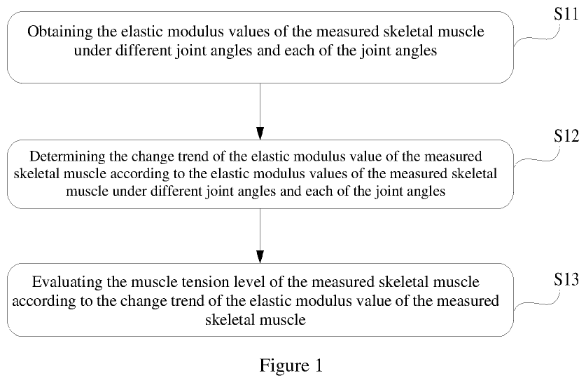

Next, the method for evaluating muscle tension disclosed in the embodiments of the application will be introduced, see FIG. 1; the method may comprise:

Step S11: obtaining the elastic modulus values of the measured skeletal muscle under different joint angles and each of the joint angles.

In the embodiment, the elastic modulus values of the measured skeletal muscle under different joint angles are measured by a measuring device, and the measured elastic modulus values of the measured skeletal muscle under different joint angles are imported into a processor. Correspondingly, the processor obtains the elastic modulus values of the measured skeletal muscle under different joint angles imported by the measuring device.

In the embodiment, the process of obtaining the elastic modulus values of the measured skeletal muscle under different joint angles may comprise:

A11. Obtaining the elastic modulus values of the measured skeletal muscle under each of the joint angles for multiple measurements.

A12. Performing an average calculation on the elastic modulus values of the measured skeletal muscle under each of the joint angles for multiple measurements, respectively, and using the result of the average calculation as the elastic modulus value of the measured skeletal muscle under the joint angle.

Using the result of the average calculation as the elastic modulus value of the measured skeletal muscle under the joint angle can improve the reliability of the elastic modulus value.

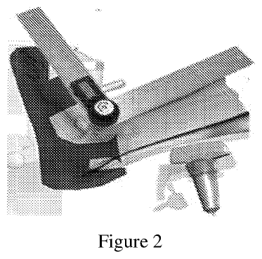

Each joint angle can be measured by a standard protractor composed of a moving arm and a fixed arm, wherein the moving arm is marked with a pointer, and the fixed arm is attached with a dial. Specifically, the joint corresponding to the measured skeletal muscle move freely or passively, and the standard protractor measures the angle of the joint corresponding to the measured skeletal muscle. During the measurement, the movable arm and the fixed arm are fixed at one end with a movable shaft, as shown in FIG. 2. The measured joint angle can be imported into the processor.

Preferably, the measuring device may be: an ultrasonic diagnostic machine.

Preferably, the process of measuring the elastic modulus values of the measured skeletal muscle under different joint angles by using an ultrasonic diagnostic machine may comprise:

determining the thickest position of muscle belly by using the B-mode imaging mode of the ultrasonic diagnostic machine to detect the cross section of the vertical muscle bundle of the measured skeletal muscle, then rotating the probe in situ, examining the long axis of the skeletal muscle along the longitudinal section in the direction of the muscle bundle at the thickest position of the muscle belly, activating the elastic imaging mode, fixing the probe position, measuring the elastic modulus value (for example, Young's modulus value) of the thickest position of muscle belly of the measured skeletal muscle continuously when the joint angle changes, and generating a dynamic ultrasound elastic image sequence.

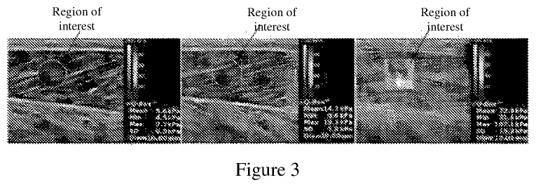

When measuring the elastic modulus value of the thickest position of muscle belly of the measured skeletal muscle, specifically, the elastic modulus value of the region of interest in the thickest position of muscle belly of the measured skeletal muscle (the elastic modulus value in the rectangular box as shown in FIG. 3) may be measured, and the average value of the elastic modulus value of the region of interest is used as the elastic modulus value of the measured skeletal muscle. Wherein the region of interest can be set to a rectangular area of 10 mm×10 mm.

It should be noted that since the thickest position of muscle belly can reflect muscle tension more accurately, measuring the elastic modulus value of the thickest position of muscle belly of the measured skeletal muscle can ensure the accuracy of the measurement result.

Furthermore, the same region of interest can be measured multiple times, and the multiple measurement results can be averaged, and the result of the average calculation can be used as the elastic modulus value of the region of interest.

Step S12. Determining the changing trend of the elastic modulus value of the measured skeletal muscle according to the elastic modulus values of the measured skeletal muscle under different joint angles and each of the joint angles.

Specifically, the elastic modulus values of the measured skeletal muscle under each joint angle can be compared, and the changing trend of the elastic modulus value of the measured skeletal muscle can be determined according to the comparison result.

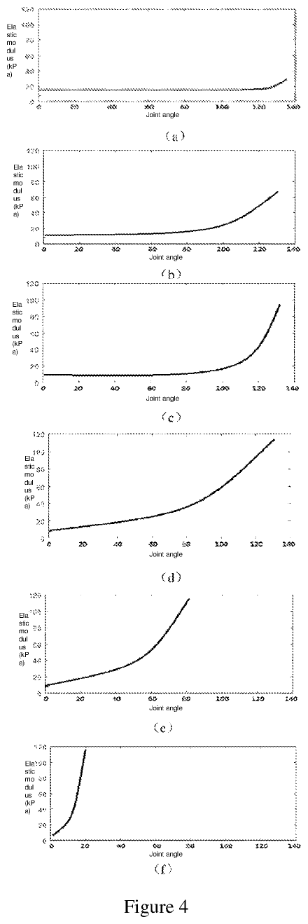

Of course, the changing trend of the elastic modulus value of the measured skeletal muscle can also be determined by using a more intuitive form. Specifically, it may be: plotting a changing trend graph of the elastic modulus value of the measured skeletal muscle with each of the joint angles as the abscissa and the elastic modulus values of the measured skeletal muscle under different joint angles as the ordinate. Wherein the changing trend graph is used to characterize the changing trend of the elastic modulus value of the measured skeletal muscle, and FIGS. 4(a)-(f) show different changing trends of the elastic modulus value of the measured skeletal muscle.

Step S13. Evaluating the muscle tension level of the measured skeletal muscle according to the changing trend of the elastic modulus value of the measured skeletal muscle.

As shown in FIG. 4(a), if the changing trend of the elastic modulus value of the measured skeletal muscle is: within the range of joint motion corresponding to each joint angle, the elastic modulus value of the measured skeletal muscle is small and increases gently, it can be determined that the muscle tension of the measured skeletal muscle is grade 0, that is, the muscle tension is low.

As shown in FIG. 4(b), if the changing trend of the elastic modulus value of the measured skeletal muscle is: within the range of joint motion corresponding to each joint angle, the elastic modulus value of the measured skeletal muscle increases slowly and increases significantly under the maximum joint angle, it can be determined that the muscle tension of the measured skeletal muscle is grade 1, that is, the muscle tension is normal.

As shown in FIG. 4(c), if the changing trend of the elastic modulus value of the measured skeletal muscle is: when the joint angle is smaller than the set angle, the elastic modulus value of the measured skeletal muscle does not change significantly with the change of the joint angle, and when the joint angle is greater than the set angle, the elastic modulus value of the measured skeletal muscle increases significantly with the change of the joint angle, it can be determined that the muscle tension of the measured skeletal muscle is grade 2, that is, the muscle tension increase slightly.

As shown in FIG. 4(d), if the changing trend of the elastic modulus value of the measured skeletal muscle is: the elastic modulus value of the measured skeletal muscle increases as the joint angle increases, and the elastic modulus value of the measured skeletal muscle increases significantly when the joint angle reaches the set angle, it can be determined that the muscle tension of the measured skeletal muscle is grade 3, that is, the muscle tension increases significantly.

As shown in FIG. 4(e), if the changing trend of the elastic modulus value of the measured skeletal muscle is: the range of the joint angle change reduces, and the elastic modulus value of the measured skeletal muscle increases significantly within the range of the joint angle change, it can be determined that the muscle tension of the measured skeletal muscle is grade 4, that is, the muscle tension increases severely.

As shown in FIG. 4(f), if the changing trend of the elastic modulus value of the measured skeletal muscle is: the range of the joint angle change is small, that is, the joint is difficult to move, and the elastic modulus value of the measured skeletal muscle increases significantly within the range of the joint angle change, it can be determined that the muscle tension of the measured skeletal muscle is grade 5, that is, the muscle tension is rigid.

In the application, the elastic modulus values of the measured skeletal muscle under different joint angles are obtained as the basis for monitoring the muscle tension of the measured skeletal muscles; determining the changing trend of the elastic modulus value of the measured skeletal muscle according to the elastic modulus values of the measured skeletal muscle under different joint angles and each of the joint angles, and evaluating the muscle tension level of the measured skeletal muscle according to the changing trend of the elastic modulus value of the measured skeletal muscle, based on the principle that the change of the elastic modulus value can characterize the change of resistance during skeletal muscle activity, and the change of resistance can reflect the situation of muscle tension.

The evaluation result of the muscle tension has important application potential for early diagnosis, pathological research, treatment evaluation, and prognosis of skeletal muscle system diseases.

Next, a device for evaluating muscle tension provided by the application will be introduced, and the device for evaluating muscle tension introduced below and the method for evaluating muscle tension introduced above can be cross-referenced.

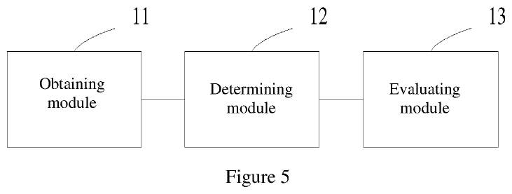

Please refer to FIG. 5, the device for evaluating muscle tension comprises: an obtaining module 11, a determining module 12, and an evaluating module 13.

Obtaining module 11 is configured to obtain the elastic modulus values of the measured skeletal muscle under different joint angles and each of the joint angles.

Determining module 12 is configured to determine the changing trend of the elastic modulus value of the measured skeletal muscle according to the elastic modulus values of the measured skeletal muscle under different joint angles and each of the joint angles.

Evaluating module 13 is configured to evaluate the muscle tension level of the measured skeletal muscle according to the changing trend of the elastic modulus value of the measured skeletal muscle.

In the embodiment, obtaining module 11 may comprise:

an obtaining submodule, configured to obtain the elastic modulus values of the measured skeletal muscle under each of the joint angles for multiple measurements.

and a determining submodule, configured to perform an average calculation on the elastic modulus values of the measured skeletal muscle under each of the joint angles for multiple measurements, respectively, and use the result of the average calculation as the elastic modulus value of the measured skeletal muscle under the joint angle.

In the embodiment, the elastic modulus values of the measured skeletal muscle under different joint angles are measured by an ultrasonic diagnostic machine.

Preferably, the elastic modulus values of the measured skeletal muscle under different joint angles are: the elastic modulus values of the thickest position of muscle belly of the measured skeletal muscle under different joint angles measured by the ultrasonic diagnostic machine.

In the embodiment, determining module 12 may comprise:

a plotting module, configured to plot a changing trend graph of the elastic modulus value of the measured skeletal muscle with each of the joint angles as the abscissa and the elastic modulus values of the measured skeletal muscle under different joint angles as the ordinate;

and the changing trend graph is used to characterize the changing trend of the elastic modulus value of the measured skeletal muscle.

It should be noted that the embodiments in the specification are all described in a progressive manner, and each embodiment focuses on the differences from other embodiments, and the same or similar parts among embodiments can be referred to each other. For the device embodiments, since they are basically similar to the method embodiments, the description is relatively simple, and for the related parts, please refer to the partial description of the method embodiments.

Finally, it should be noted that relational terms such as first and second herein are only used to distinguish one entity or operation from another entity or operation, and do not necessarily require or imply any such actual relationship or order between these entities or operations. Moreover, the terms “include”, “comprise” or any other variants thereof are intended to cover non-exclusive inclusion, so that a process, method, article or device including a series of elements not only includes those elements, but also includes other elements that are not explicitly listed, or elements inherent to such process, method, article, or device. If there are no more restrictions, the element defined by the sentence “including a . . . ” does not exclude the existence of other same elements in the process, method, article, or device that includes the element.

For the convenience of description, the device is divided into various units by function and described separately. It is without doubt that when implementing the application, the functions of each unit can be implemented in the same or multiple software and/or hardware.

From the description of the above implementation manners, it can be known that those skilled in the art can clearly understand that the application can be implemented by means of software plus a necessary general hardware platform. Based on such understanding, the technical solution of the application essentially or the part that contributes to the prior art may be embodied in the form of a software product, and the computer software product may be stored in a storage medium, such as ROM/RAM, magnetic disk, CD-ROM, etc., including several instructions to make a computer device (which may be a personal computer, a server, or a network device, etc.) execute the methods described in the embodiments or some parts of the embodiments of the application.

The above provides a detailed introduction to the method and device for evaluating muscle tension provided by the application. Specific examples are used herein to illustrate the principles and implementations of the application, and the description of the above examples is only used to help understand the method and core idea of the application; meanwhile, for those of ordinary skill in the art, there will be changes in the specific implementation and the scope of application according to the idea of the application. In summary, the content of the specification should not be construed as a limitation on the application.

1. A method for evaluating muscle tension, comprising:
obtaining the elastic modulus values of the measured skeletal muscle under different joint angles and each of the joint angles;
determining the changing trend of the elastic modulus value of the measured skeletal muscle according to the elastic modulus values of the measured skeletal muscle under different joint angles and each of the joint angles;
and evaluating the muscle tension level of the measured skeletal muscle according to the changing trend of the elastic modulus value of the measured skeletal muscle.

2. The method as specified in claim 1, wherein obtaining the elastic modulus values of the measured skeletal muscle under different joint angles comprises:
obtaining the elastic modulus values of the measured skeletal muscle under each of the joint angles for multiple measurements;
performing an average calculation on the elastic modulus values of the measured skeletal muscle under each of the joint angles for multiple measurements, respectively, and using the average calculation as the elastic modulus value of the measured skeletal muscle under the joint angle.

3. The method as specified in claim 1, wherein the elastic modulus values of the measured skeletal muscle under different joint angles are measured by an ultrasonic diagnostic machine.

4. The method as specified in claim 3, wherein the elastic modulus values of the measured skeletal muscle under different joint angles are: the elastic modulus values of the thickest position of muscle belly of the measured skeletal muscle under different joint angles measured by the ultrasonic diagnostic machine.

5. The method as specified in claim 1, wherein determining the changing trend of the elastic modulus value of the measured skeletal muscle according to the elastic modulus values of the measured skeletal muscle under different joint angles and each of the joint angles comprises:
plotting a changing trend graph of the elastic modulus value of the measured skeletal muscle with each of the joint angles as the abscissa and the elastic modulus values of the measured skeletal muscle under different joint angles as the ordinate;
and the changing trend graph is used to characterize the changing trend of the elastic modulus value of the measured skeletal muscle.

6. A device for evaluating muscle tension comprising:
an obtaining module, configured to obtain the elastic modulus values of the measured skeletal muscle under different joint angles and each of the joint angles;
a determining module, configured to determine the changing trend of the elastic modulus value of the measured skeletal muscle according to the elastic modulus values of the measured skeletal muscle under different joint angles and each of the joint angles;
and an evaluating module, configured to evaluate the muscle tension level of the measured skeletal muscle according to the changing trend of the elastic modulus value of the measured skeletal muscle.

7. The device as specified in claim 6, wherein the obtaining module comprises:
an obtaining submodule, configured to obtain the elastic modulus values of the measured skeletal muscle under each of the joint angles for multiple measurements;
and a determining submodule, configured to perform an average calculation on the elastic modulus values of the measured skeletal muscle under each of the joint angles for multiple measurements, respectively, and use the result of the average calculation as the elastic modulus value of the measured skeletal muscle under the joint angle.

8. The device as specified in claim 6, wherein the elastic modulus values of the measured skeletal muscle under different joint angles are measured by an ultrasonic diagnostic machine.

9. The device as specified in claim 8, wherein the elastic modulus values of the measured skeletal muscle under different joint angles are: the elastic modulus values of the thickest position of muscle belly of the measured skeletal muscle under different joint angles measured by the ultrasonic diagnostic machine.

10. The device as specified in claim 6, wherein the determining module comprises:
a plotting module, configured to plot a changing trend graph of the elastic modulus value of the measured skeletal muscle with each of the joint angles as the abscissa and the elastic modulus values of the measured skeletal muscle under different joint angles as the ordinate;
and the changing trend graph is used to characterize the changing trend of the elastic modulus value of the measured skeletal muscle.
